(19) United States
(12) Patent Application Publication
(10) Pub. No.: US 2021/0132172 A1
(43) Pub. Date: May 6, 2021

(54) COMPOSITION FOR REMOVING NOISE FROM MRI AND PAD USING THE SAME

(71) Applicant: Jung-Eun Baek, Seoul (KR)

(72) Inventor: Kyung Seok PARK, Goyang-si, Gyeonggi-do (KR)

(73) Assignee: Jung-Eun BAEK, Seoul (KR)

(21) Appl. No.: 16/492,082

(22) PCT Filed: Mar. 8, 2018
(86) PCT No.: PCT/KR2018/002757

§ 371 Date: Sep. 6, 2019

(30) Foreign Application Priority Data
Mar. 9, 2017 (KR) 10-2017-0030077
May 16, 2017 (KR) 10-2017-0060364

(51) Int. Cl. G01R 33/565; A61B 5/00; A61B 5/055

(52) U.S. Cl. CPC G01R 33/56536; A61B 5/055; A61B 5/7203

(57) ABSTRACT

Provided are a composition for removing noise from an MRI, including a compound having a hydrogen bond, and a pad using the same, wherein the composition is applied in a non-invasive way causing no side effects in the human body, the composition is not toxic and thus is safe, the composition is used in the manner of covering an area requiring diagnosis or being attached to an MRI apparatus, and thus has the advantages of improving images of a relatively wide range, and obtaining more accurate image information than restoration of a distorted image by a program, and the composition is expected to be easily commercialized by companies because the costs required to manufacture the composition are low.

### TECHNICAL FIELD

The present invention relates to a composition for removing noise from MRI and a pad using the same, in which the composition includes a compound having a functional group capable of hydrogen bonding.

### BACKGROUND ART

A common MRI contrast agent is injected into the human body by an invasive procedure to enhance a signal according to the resonance of the nucleus of a hydrogen atom in the body, and thus obtain a clearer image of the core part of the body. However, the injection of a contrast agent by an invasive procedure, for example, intravenous injection, causes mild symptoms that need no special treatment, such as vomiting, abdominal pain, diarrhea, high blood pressure, headache, fever, chills, coughing, nasal congestion, etc., and also serious side effects such as dizziness, low blood pressure, bradycardia, systemic urticaria, angioedema, dyspnea, nephrogenic fibrosing dermopathy, nephrogenic systemic fibrosis (NSF), etc., and if there is a material affecting a magnetic field, such as a metal implant in the body, there is a limitation in that noise generated in an image is not removed.

In addition, the technique of removing noise due to a metal implant imbedded in the body, which is not able to be solved by a contrast agent, is mainly to estimate and compensate the defective part of the image using a computer algorithm, and this technique has problems of inaccuracy and insufficiently wide reproducible part. To solve the above-described problems, while much research is progressing to solve metal material noise and non-uniform distorted images, which are generated in the process of obtaining human body images, such that normal tissue and diseased tissue are more clearly distinguished using an MRI image (Korean Patent No. 10-1262479), there is less research on image enhancement technology that can improve both the limitation of image enhancement by software and the side effect of a contrast agent which is generated in the body. Particularly, no technology using a non-invasive composition, which is not injected into the body, in order to improve the above-described problems has been reported yet.

The inventors have intensively attempted to remove noise generated in MRI image acquisition by a non-invasive method different from a common contrast agent, thereby confirming that metal noise can be effectively removed by attaching a material including a functional group capable of hydrogen bonding to one region of a human body requiring diagnosis or by a method of covering the region with a pad including the material, and also confirming that the same effect can be obtained when the composition or pad of the present invention is attached to or inserted into an MRI coil.

Thus, the present invention was completed.

### DISCLOSURE

### Technical Problems

Therefore, to solve the above-described problems, the inventors have intensively attempted to remove noise when an MRI image is obtained, and thereby confirmed that noise caused by a metal material can be effectively removed and image quality can be improved by restoring noise in MRI images and phantom images using a material having a functional group capable of hydrogen bonding. Thus, the present invention was completed.

Accordingly, the present invention is directed to providing a composition for removing noise from an MRI image, which includes a compound having a functional group capable of hydrogen bonding.

The present invention is also directed to providing a pad for removing noise from an MRI image using the composition.

The present invention is also directed to providing a coil for an MRI apparatus into which the composition is injected.

However, technical problems to be solved in the present invention are not limited to the above-described problems, and other problems which are not described herein will be fully understood by those of ordinary skill in the art from the following description.

### Technical Solutions

One aspect of the present invention provides a composition for removing noise from an MRI image, which includes a compound having a functional group capable of hydrogen bonding.

The composition may improve the quality of an MRI image, but the present invention is not limited thereto.

In addition, the compound may be one or more selected from the group consisting of water (H₂O), amyl alcohol (C₅H₁₁OH), butyl alcohol (CH₃CH₂CH₂OH), ethanol (C₂H₅OH), methyl alcohol (CH₃OH), ethylene glycol (OHCH₂CH₂OH), formaldehyde (HCHO), nitrobenzene (C₆H₅NO₂), propylene glycol (OH(CH₂)₃OH), hydrogen peroxide (H₂O₂), acetic acid (CH₃COOH), acetic anhydride (C₄H(O₃), butanoic acid (C₃H₇COOH), benzenesulfonic acid (C₆H₅SO₃H), benzoic acid (C₆H₅COOH), chromic acid (H₂CrO₄), citric acid (C₆H₈O₇H₂O), hexafluorosilicic acid (H₂SiF), fluoroboric acid (HBF₄), formic acid (HCOOH), hydrochloric acid (HCl), hydrocyanic acid (HCN), hydrofluoric acid (HF), hypochlorous acid (HClO), lactic acid (CH₃CHOHCOOH), maleic acid ((CHOOH)₂), nitric acid (HNO₃), oleic acid (C₁₈H₃₄O₂), phosphoric acid (H₃PO₄), perchloric acid (HClO₄), sulfuric acid (H₂SO₄), stearic acid (C₁₇H₃₅COOH), tnchloroacetic acid (CCl₃COOH), ammonia (NH₄OH), sodium hydrogen carbonate (NaHCO₃), ammonium carbonate ((NH₄)₂CO₃), ammonium chloride ((NH₄)₂Cl), ammonium nitride (NH₄NO₃), ammonium persulfate ((NH₄)₂S₂O₈), glycerin (C₃H₈O₃), ammonium sulfate ((NH₄)₂SO₄) and ammonium bicarbonate (NH₄HCO₃), but the present invention is not limited thereto.

In addition, the water may be any one selected from the group consisting of light water, heavy water, tritiated water, and distilled water, but the present invention is not limited thereto.

In addition, the compound having the hydrogen bond may be contained at 40 wt % or more with respect to the total weight of the composition, but the present invention is not limited thereto.

In addition, the composition of the present invention may further include a material that can be used as a contrast agent to enhance an image, and the material that can be used as a contrast agent may be selected from the group consisting of gadoxetic acid disodium, calcium oxide, trometamol, sodium hydroxide, hydrochloric acid, an injection solution, calteridol calcium, gadolinium oxide, calcium, meglumine, EOB DTPA, gadoteridol and a chelating agent, but the present invention is not limited thereto.

In addition, the noise may be generated by a metal inserted into the body.

In addition, the composition of the present invention may be charged in a pad, and not only may the composition of the present invention be used as a pad for covering the body, but it may also be attached/detached to a main body coil, a shim coil, an RF coil, an array coil, a surface coil, a spine coil, a head coil, an extremity coil or a torso coil for an MRI apparatus.

In addition, the composition of the present invention may be used in the manner of being injected into a main body coil, a shim coil, an RF coil, an array coil, a surface coil, a spine coil, a head coil, an extremity coil or a torso coil for an MRI apparatus.

Another aspect of the present invention provides a pad for removing noise from an MRI image, which includes the composition for removing noise from an MRI image.

The pad may improve the quality of MRI images, but the present invention is not limited thereto.

The pad of the present invention may further include a cover accommodating the composition. The cover may be a vinyl cover, but the present invention is not limited thereto.

In addition, the pad of the present invention may be a pad that can be transformed into a shape of the human body, for example, the head, limbs, spine or abdomen.

In addition, the pad of the present invention may be a pad capable of being attached/detached to a main body coil, a shim coil, an RF coil, an array coil, a surface coil, a spine coil, a head coil, an extremity coil or a torso coil for an MRI apparatus, but the present invention is not limited thereto.

In addition, the pad may further include a filler, which may be a liquid gel form or a solid form. When the filler is a solid form, it may be a low-fluidic bead or powder type, or a combination of beads and a gel.

Still another aspect of the present invention provides a coil for an MRI apparatus into which the composition for removing noise from an MRI image is injected.

The coil for an MRI apparatus may be one or more types of coils selected from the group consisting of a main body coil, a shim coil, an RF coil, an array coil, a surface coil, a spine coil, a head coil, an extremity coil, and a torso coil, but the present invention is not limited thereto.

Yet another aspect of the present invention provides a method of removing noise from an MRI image using the composition including the compound having a functional group capable of hydrogen bonding.

Yet another aspect of the present invention provides a method of obtaining an MRI image reduced in noise, which includes: manufacturing a pad including a compound having a functional group capable of hydrogen bonding; attaching the pad to a desired region of a subject; and obtaining an MRI image in the desired region using an MRI apparatus.

Yet another aspect of the present invention provides a use of the compound having a functional group capable of hydrogen bonding for removing noise from an MRI image.

### Advantageous Effects

A compound having a functional group capable of hydrogen bonding of the present invention has advantages of naturally generating a high signal through indirect energy exchange according to the magnetization of a hydrogen bond, and activating an image of tissue which is not properly embodied due to noise generated by a metal. A damaged tissue image due to a metallic material is theoretically impossible to reproduce and recover, but a pad according to the present invention makes capturing the defected image possible and thus the image defect problem can be fundamentally solved. According to imaging technology, an image that is theoretically difficult to recover can be reproduced realistically. Also, the present invention is completed based on fundamental and concrete scientific knowledge, for example, a basic theory of MRI for obtaining a basic image, in which an organ of the human body is visualized according to the magnetic susceptibility of hydrogen, demonstrating an effect of removing noise caused by a metallic material and the improvement in image quality based on this theory.

In addition, since the composition according to the present invention can be applied by a non-invasive method, unlike an MRI contrast agent, it has no problem causing a side effect in the human body, no toxicity and this is safe. Moreover, the composition of the present invention is used in the manner of covering a region in need of diagnosis or being attached to an MRI apparatus, such that there are advantages of improving a relatively wide range of images, and obtaining more accurate image data than that obtained when distorted images are restored by software using an algorithm. At the same time, the composition of the present invention has a low price, such that it can be used as an effective and economical auxiliary means for improving an MRI image.

### DESCRIPTION OF DRAWINGS

FIG. 1A shows fruit juice acetic acid, a thickening agent and a glycerin (ultrasound gel) solution for manufacturing an acetic acid pad, and FIG. 1B shows a gel prepared by mixing the above-described materials.

FIG. 2 shows an acetic acid gel pad manufactured by containing mixed gel-type fruit juice acetic acid in a sanitary vinyl-type container.

FIG. 3 shows a phantom manufactured by adding a clip to a fat container.

FIG. 4A shows the result of comparing ROI mean values of a general phantom image, a water pad and an acetic acid pad, and FIG. 4B shows the comparison experiment result for a decrease in noise caused by a metallic material, showing the difference between a GE Healthcare auxiliary pad (left panel of FIG. 4B) and an acetic acid pad (right panel of FIG. 4B).

FIGS. 5A and 5B show the result of comparing an image with noise caused by a metallic material before (left panels of FIGS. 5A and 5B) and after (right panels of FIGS. 5A and 5B) the use of a pad, FIG. 5C shows the result of comparing phantom images according to the difference in magnetic susceptibility of the human body, and FIGS. 5D to 5L show the results of visually observing the improvement in noise caused by a metallic material and an image quality-improving effect before (left panels of FIGS. 5D to 5L) and after (right panels of FIGS. 5D to 5L) the use of a pad.

FIG. 6 shows the result of confirming the effect of removing noise from an MRI image using water and acetic acid.

FIG. 7 shows the result of confirming the effect of removing noise from an MRI image using citric acid.

FIG. 8 shows the result of confirming the effect of removing noise from an MRI image using sodium hydrogen carbonate.

FIG. 9 shows the result of confirming the effect of removing noise from an MRI image using ethanol.

FIG. 10 shows the result of confirming the effect of removing noise from an MRI image using glycerin.

### MODES OF THE INVENTION

Hereinafter, the present invention will be described in detail with reference to the following examples or the accompanying drawings. However, the examples or the description of the drawings merely explain exemplary embodiments of the present invention, and the scope of the present invention is not intended to be limited or narrowly interpreted by the description of the present invention.

### [Example 1] Preparation and Methods of Experiment

1-1. Manufacture of the Acetic Acid Pad

Since acetic acid is easy to purchase and harmless to the human body and to investigate whether the removal of noise from an MRI image and an image quality-improving effect can be obtained using the acetic acid, a pad was manufactured using a naturally obtained concentration of acetic acid prepared from fruit juice.

A small amount of glycerin was mixed with fruit juice acetic acid, and a thickening agent was used to easily attach the mixture to a patient's implant site. Here, the used thickening agent refers to a mixture of glycerin (ultrasound gel component) and a common thickening component, and the thickening agent can be replaced with an ultrasound gel.

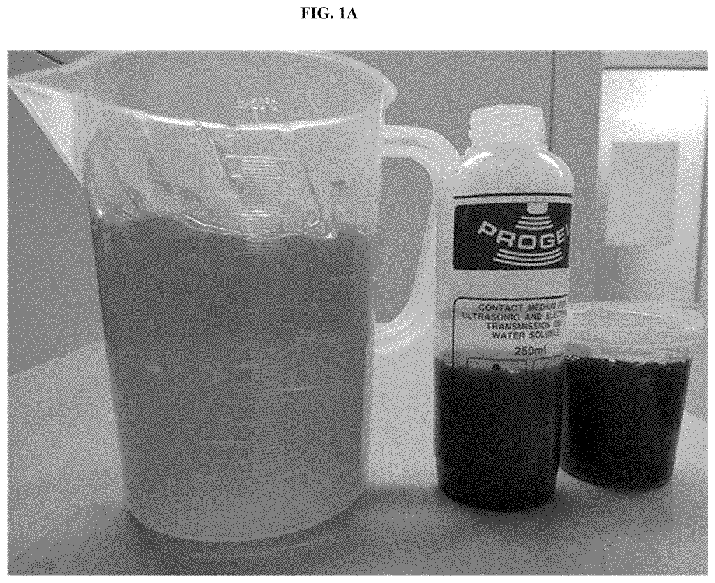

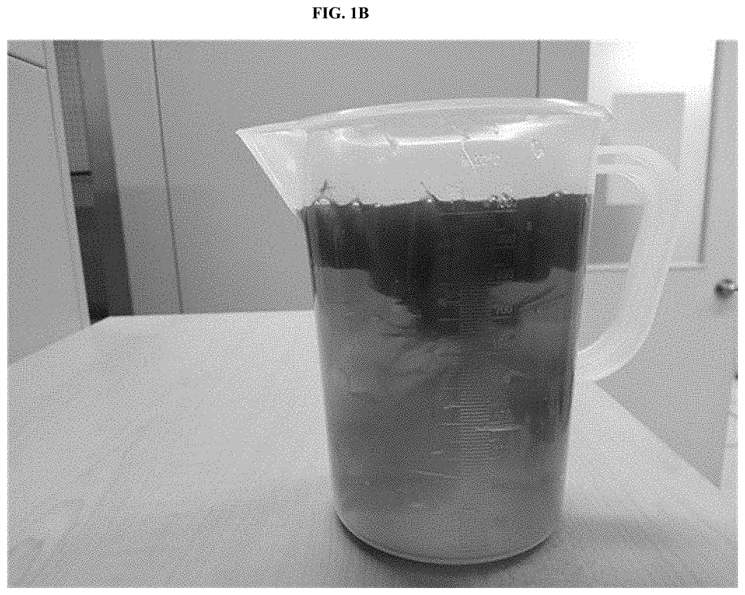

As shown in FIG. 1A, about 1 L of fruit juice acetic acid, a thickening agent and a glycerin (ultrasound gel) solution were prepared, and then they are mixed in a ratio of 200 mL:800 mL (the fruit juice acetic acid:a mixture of a thickening agent and glycerin (ultrasound gel)) until the mixture becomes viscous, thereby preparing a gel (see FIG. 1B).

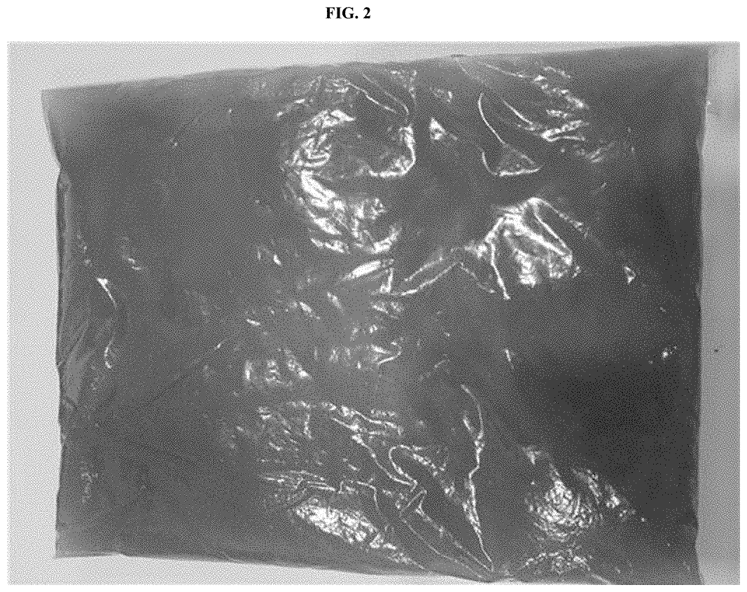

Afterward, as shown in FIG. 2, the mixed gel-type fruit juice acetic acid was contained in a sanitary vinyl-type container and stored at room temperature (15 to 25° C.), and then a test was performed once by attaching the acetic acid gel pad to the periphery of the implant at the final stage of the test for a patient who has given verbal consent.

1-2. Comparison Experiment Before/after Use of Acetic Acid Pad

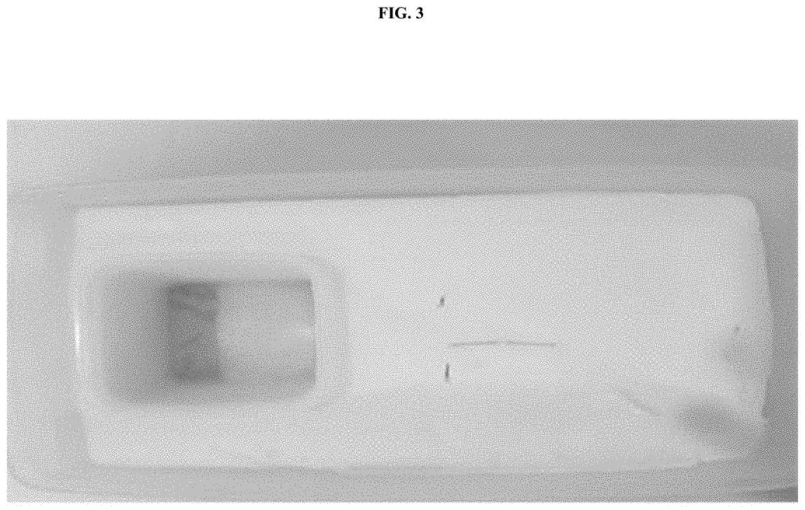

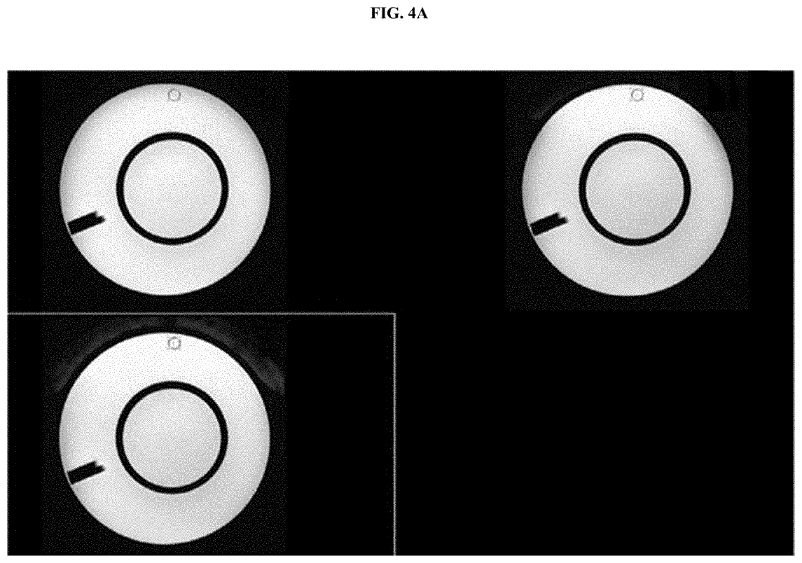

To overcome the limits of a human body experiment, as shown in FIG. 3, using a phantom manufactured by adding a clip to a fat container, a comparison experiment was sequentially performed on various pads, for example, a common phantom, a motion-preventing pad, a sandbag pad, a rice pad (tissue equivalent), a GE Healthcare abdominal fat-reducing pad, water, and an acetic acid pad.

More specifically, using specified protocol T1 Fat Saturation (Fov 240×207, TR 750 ms, TE 12 ms, Thickness 4 mm, Gap 1.5 mm), an international standard phantom was used as a target, and observation was performed before and after the use of a fruit juice acetic acid component pad. After the test, images of the region of interest (ROI) sites were obtained using MacroView (INFINITT) and a workstation ROI (contrast HU-value; M-view v 5.4.10.61).

After the phantom experiment, a patient observation test was performed before and after the use of the acetic acid pad, and thus images were obtained.

### [Example 2] Confirmation of Experiment Results for Phantoms Before/after Use of Acetic Acid Pad

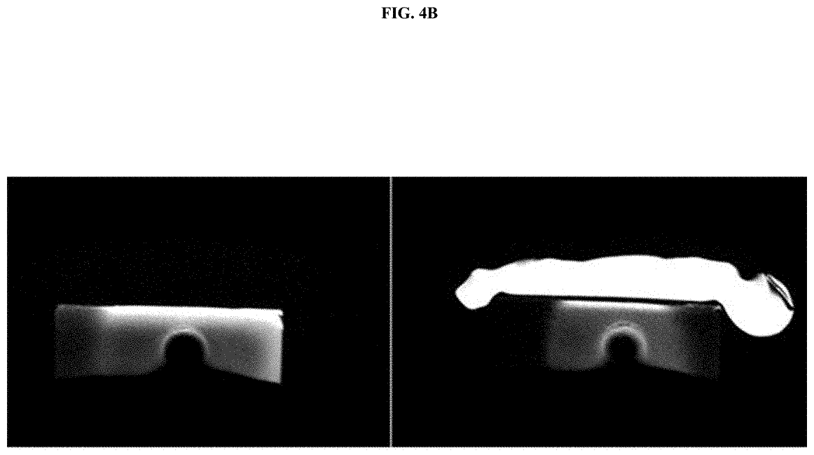

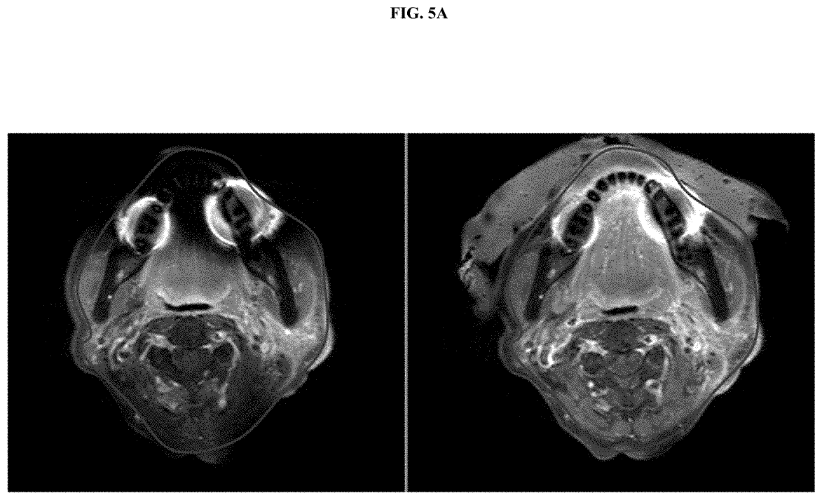

For international standard quality control (QC), that is, checking of the improvement of apparatus quality, an experiment was performed using the international standard QC phantom according to the method described in Example 1-2, and then a common phantom image, and the ROI mean values of a water pad and an acetic acid pad were compared. As a result, as shown in FIG. 4A and Table 1, the mean value of the non-mounted common phantom image was measured at 2029 (upper left panel of FIG. 4A), the mean value of the mounted water pad was measured at 2318 (the upper right panel of FIG. 4A), and the mean value of the acetic acid pad was measured at 2364 (lower panel of FIG. 4A).

TABLE 1

| Type | ROI mean value |
|---|---|
| Common phantom | 2029 |
| Water pad | 2318 |
| Acetic acid pad | 2364 |

In addition, as shown in FIG. 4B, the comparison experiment for the reduction in noise caused by a metallic material showed a difference in images of the GE Healthcare auxiliary pad (left panel of FIG. 4B) and the acetic acid pad (right panel of FIG. 4B). From the above result, it can be confirmed that the higher the acetic acid content, the higher the ROI mean value, and thus the noise reduction rate was increased. Accordingly, it can be seen that acetic acid reduces the noise caused by a metallic material, demonstrating that the higher the hydrogen content and the higher the concentration of hydrogen, the higher the ROI measured value and noise-reducing effect.

### [Example 3] Confirmation of Experiment Results for Patients Before/after Use of Acetic Acid Pad

To compare the images with noise caused by a metallic material before and after the use of an acetic acid pad, patients were observed according to the method described in Example 1-2. Here, to compare the objective measurement data on the effect of using an acetic acid pad, the ROI mean value of the minimum and maximum standard deviations in an image quality contrast was compared.

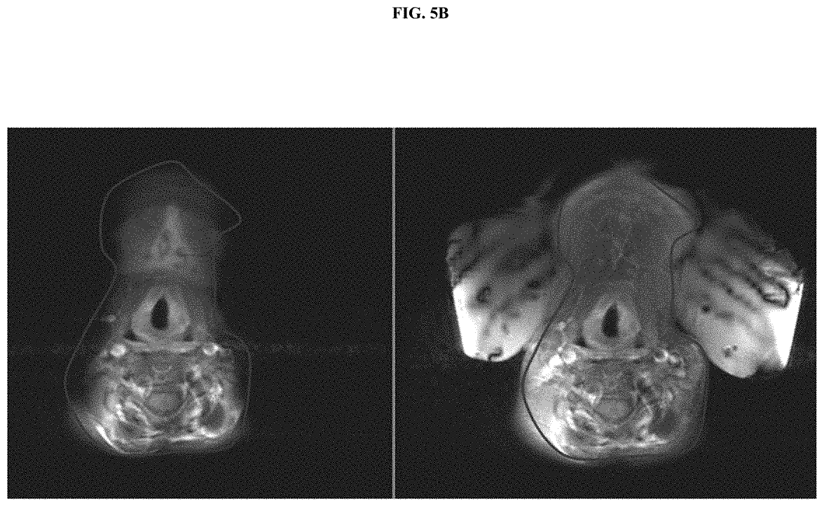

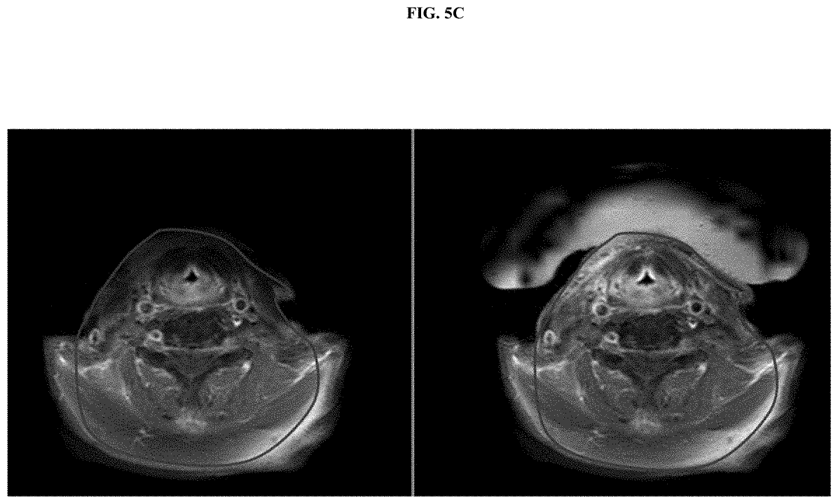

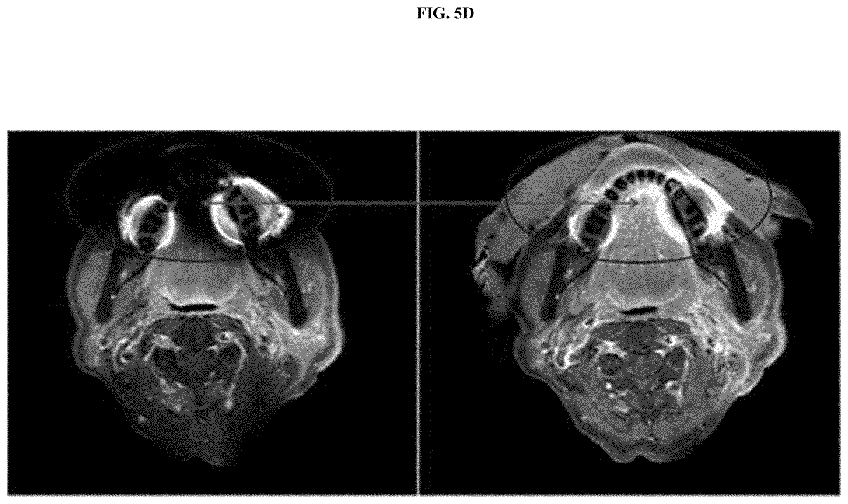

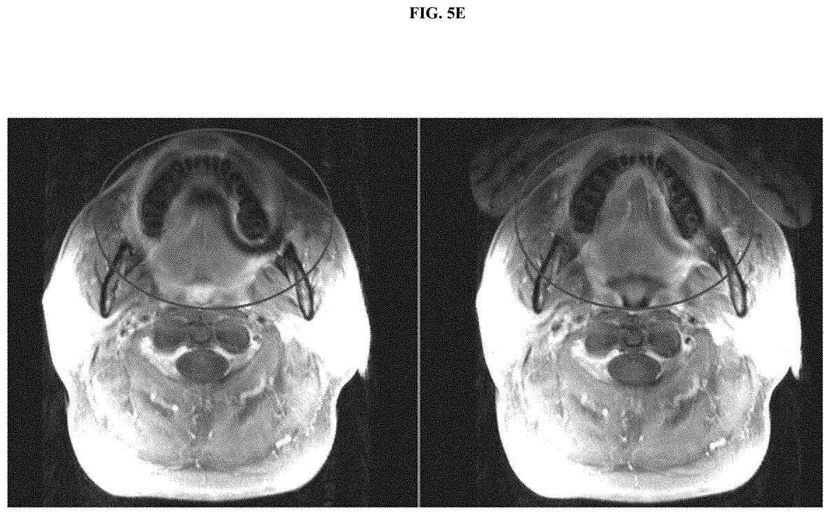

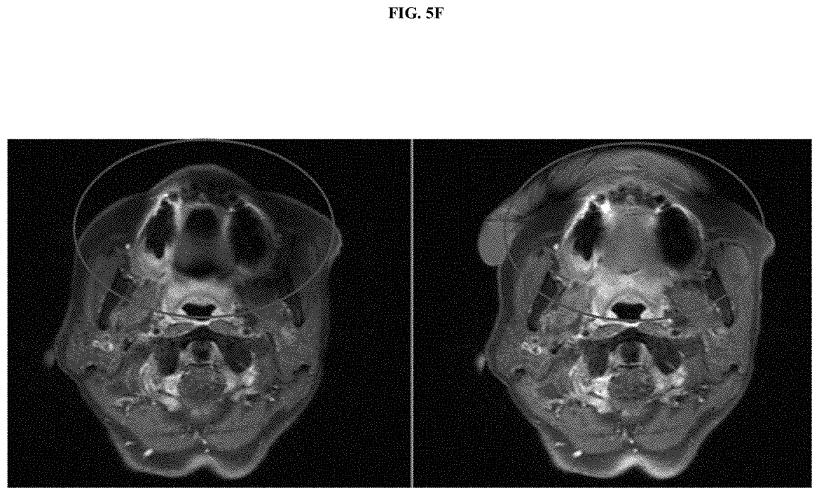

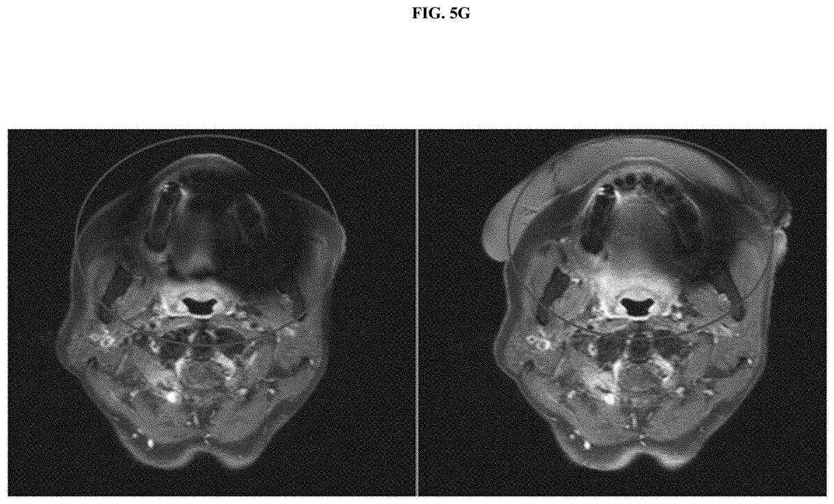

As a result, as shown in FIGS. 5A to 5C and Table 2, comparing the objective ROI mean values before and after the use of an acetic acid pad, the RO mean value after the use of the pad (right panels of FIGS. 5A to 5C), compared with before the use of the pad (left panels of FIGS. 5A to 5C), were all increased, demonstrating that the image contrast after the use of an acetic acid pad is superior.

TABLE 2

ROI mean value

| | Before pad use | After pad use |
|---|---|---|
| Drawing | (left panel of a drawing) | (right panel of a drawing) |
| FIG. 5A | 380 | 533 |
| FIG. 5B | 462 | 599 |
| FIG. 5C | 852 | 1011 |

Moreover, FIG. 5C shows the result of comparing phantom images according to the difference in magnetic susceptibility of the human body, illustrating that the use of acetic acid pads (right panel of FIG. 5C) in an area of the cancer lesion of the neck, which is not shown in the phantom image (left panel of FIG. 5C), greatly improved the image quality. In addition, as shown in FIGS. 5D to 5L, since visual observation can show that the improvement in noise caused by a metallic material and the image quality improvement effect are obvious after the pad use (right panels of FIGS. 5D to 5L) compared with that before the pad use (left panels of FIGS. 5D to 5L), in MRI images, it can be deduced that the information of the MRI diagnosed region can be very broad using an acetic acid pad.

### [Example 4] Confirmation of the Effect of Removing Noise from MRI Image of the Pad Including a Compound Having a Functional Group Capable of Hydrogen Bonding

As shown in Example 3, human images were obtained before and after the use of a pad consisting of acetic acid as a component, and to confirm the MRI image noise-removing effect of each pad filled with citric acid, sodium hydrogen carbonate, ethanol or glycerin, which has a high hydrogen content, for example, acetic acid, a region of interest (ROI) mean value in the image was measured using the phantom manufactured herein.

An acetic acid, citric acid, sodium hydrogen carbonate, ethanol or glycerin concentration of 0% represents water (H₂O₂), and an MRI image was obtained by targeting a component containing hydrogen, as a criterion for image acquisition, the natural state, water (H₂O), has been standardized according to MRI periodic inspection and international standard inspection.

4-1. Confirmation of Removal of Noise from MRI Image Using Water (H₂O) and Acetic Acid

According to the method described in Example 1-1, a pad was manufactured by filling a pad with acetic acid (CH₃COOH), and an MRI image was measured.

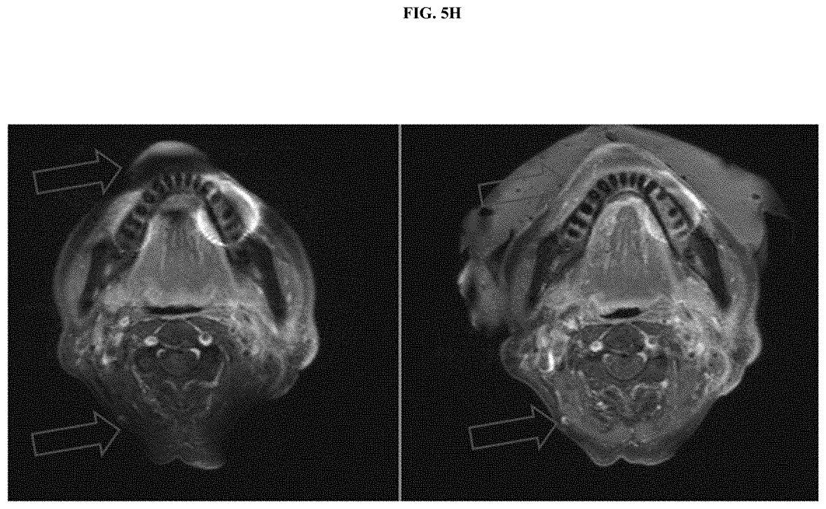

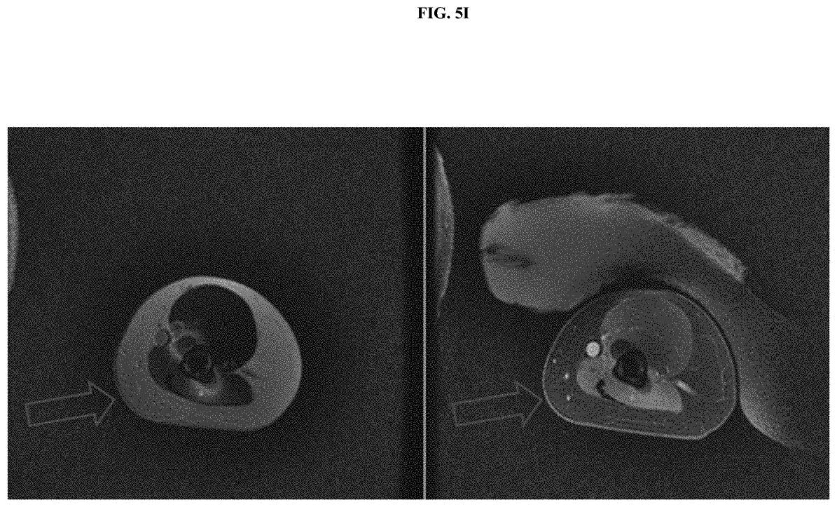

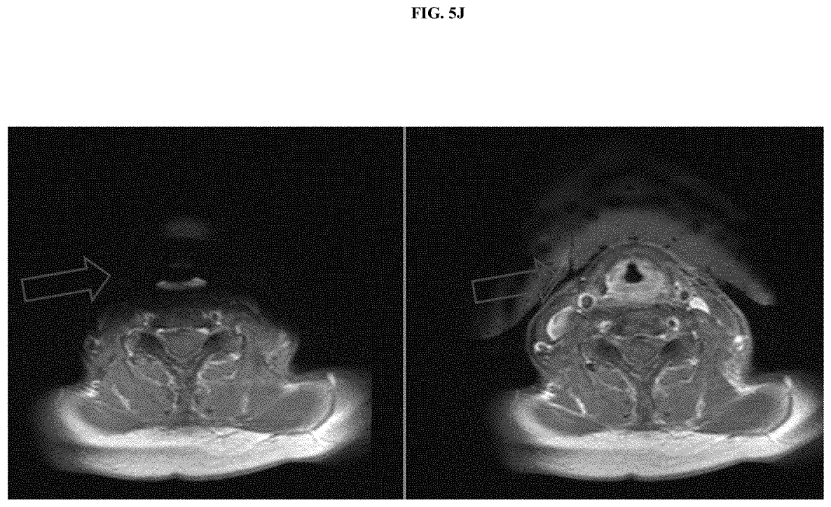

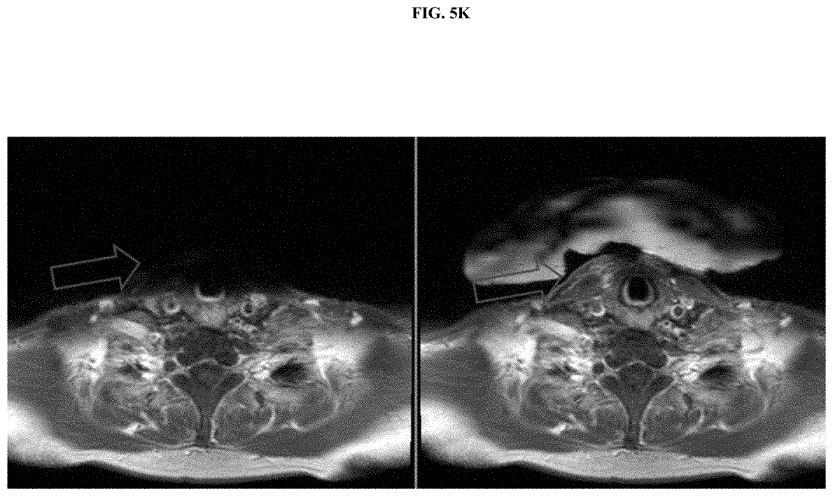

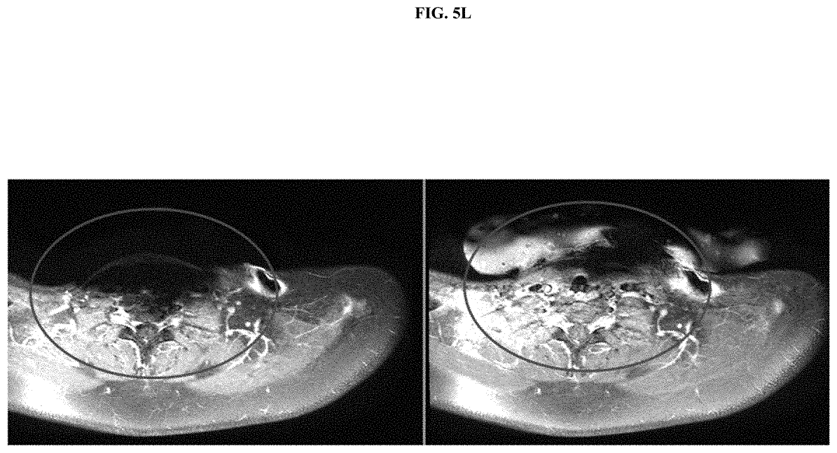

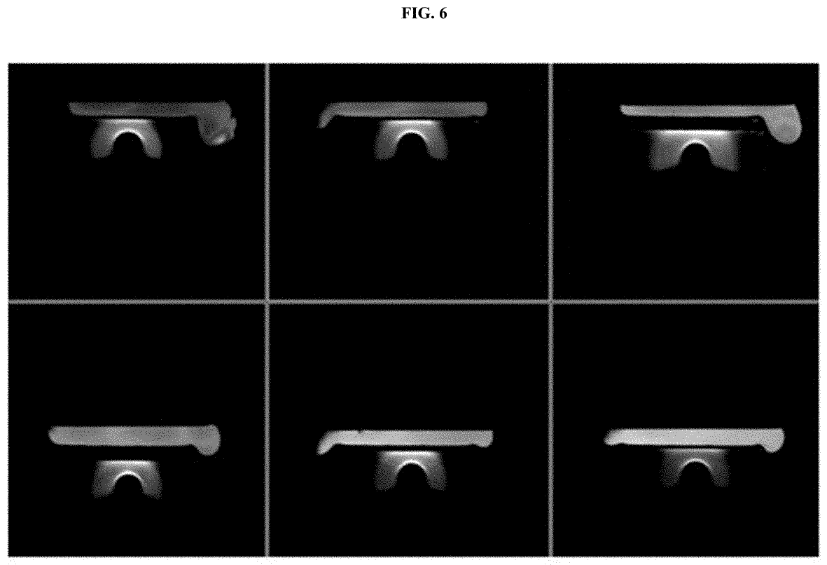

As a result, as shown in FIG. 6 and Table 3, it can be observed that, from the left side to the right side of FIG. 6 (the concentration of acetic acid in the upper panels was sequentially increased to 20% and 40% from 0% (H₂O), and similarly, the concentration of acetic acid in the lower panels was sequentially increased to 60%, 80% and 100% from the left side to the right side of the drawing), as the concentration of acetic acid was gradually increased, the ROI mean value was increased, and the noise reduction rate of an MRI image was increased.

TABLE 3

| Concentration of acetic acid (CH₃COOH) | ROI mean value (Mean) |
|---|---|
| 0% (H₂O) | 1119 |
| 20% | 1147 |
| 40% | 1798 |
| 60% | 1868 |
| 80% | 1935 |
| 100% | 2004 |

4-2. Confirmation of Removal of Noise from MRI Image Using Citric Acid

In addition, according to the method described in Example 1-1, a pad was manufactured by filling a pad with citric acid (C₆H₈O₇H₂O), and an MRI image was measured.

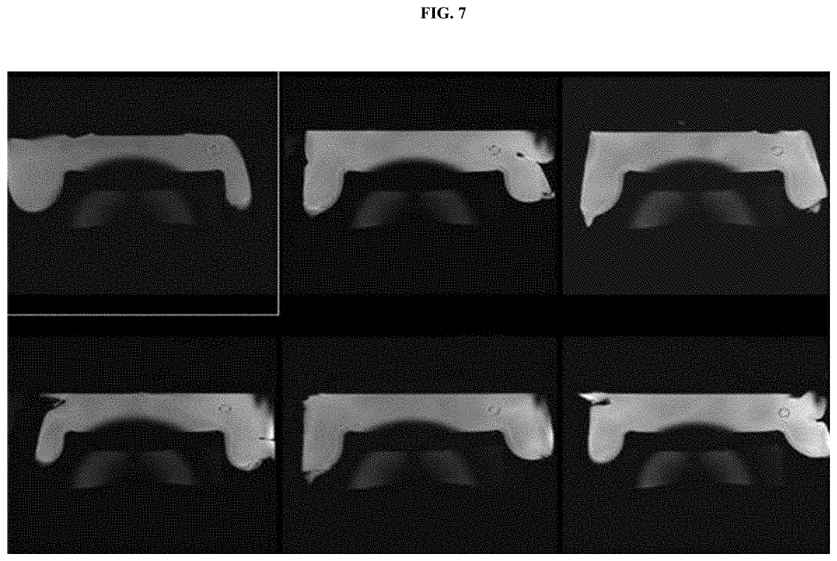

As a result, as shown in FIG. 7 and Table 4, it can be observed that, from the left side to the right side of FIG. 7 (the concentration of citric acid in the upper panels was sequentially increased to 20% and 40% from 0% (H₂O), and similarly, the concentration of citric acid in the lower panels was sequentially increased to 60%, 80% and 100% from the left side to the right side of the drawing), as the concentration of citric acid was gradually increased, the ROI mean value was increased, and the noise reduction rate of an MRI image was increased.

TABLE 4

| Concentration of citric acid (C₆H₈O₇H₂O) | ROI mean value (Mean) |
|---|---|
| 0% (H₂O) | 1480 |
| 20% | 1698 |
| 40% | 1702 |
| 60% | 1734 |
| 80% | 1762 |
| 100% | 1797 |

4-3. Confirmation of Removal of Noise from MRI Image Using Sodium Hydrogen Carbonate

According to the method described in Example 1-1, a pad was manufactured by filling a pad with sodium hydrogen carbonate (NaHCO₃), and an MRI image was measured.

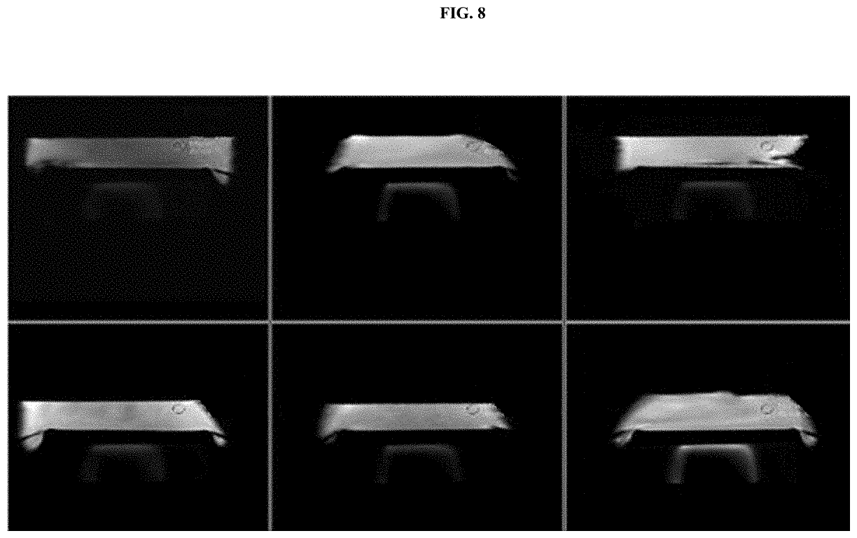

As a result, as shown in FIG. 8 and Table 5, it can be observed that, from the left side to the right side of FIG. 8 (the concentration of sodium hydrogen carbonate in the upper panels was sequentially increased to 20% and 40% from 0% (H₂O), and similarly, the concentration of sodium hydrogen carbonate at the lower panels was sequentially increased to 60%, 80% and 100% from the left side to the right side of the drawing), as the concentration of sodium hydrogen carbonate was gradually increased, the ROI mean value was increased, and the noise reduction rate of an MRI image was increased.

TABLE 5

| Concentration of sodium hydrogen | ROI mean value |
|---|---|
| carbonate (NaHCO₃) | (Mean) |
| 0% (H₂O) | 1216 |
| 20% | 1439 |
| 40% | 1653 |
| 60% | 1622 |
| 80% | 1734 |
| 100% | 1730 |

4-4. Confirmation of Removal of Noise from MRI Image Using Ethanol

According to the method described in Example 1-1, a pad was manufactured by filling a pad with ethanol (C₂H₅OH), and an MRI image was measured.

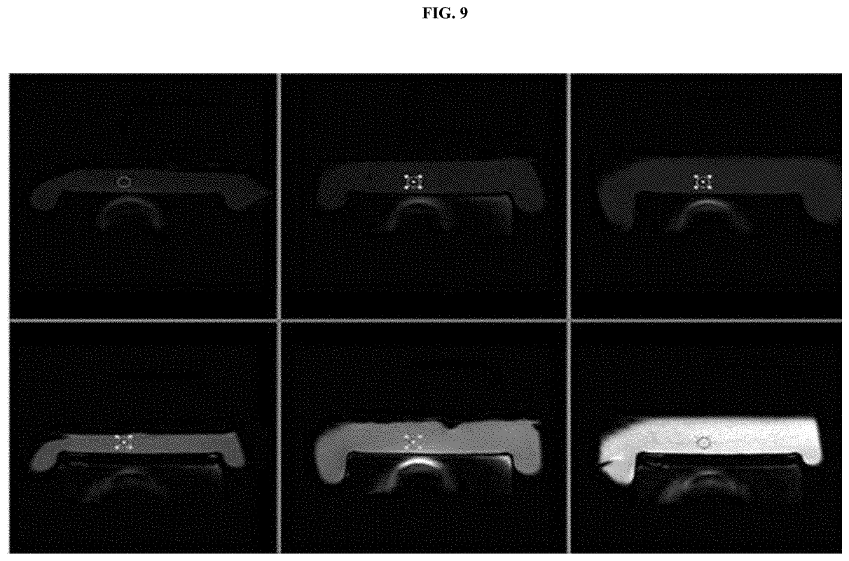

As a result, as shown in FIG. 9 and Table 6, it can be observed that, from the left side to the right side of FIG. 9 (the concentration of ethanol in the upper panels was sequentially increased to 20% and 40% from 0% (H₂O), and similarly, the concentration of ethanol in the lower panels was sequentially increased to 60%, 80% and 100% from the left side to the right side of the drawing), as the concentration of ethanol was gradually increased, the ROI mean value was increased, and the noise reduction rate of an MRI image was increased.

TABLE 6

| Concentration of ethanol (C₂H₅OH) | ROI mean value (Mean) |
|---|---|
| 0% (H₂O) | 1519 |
| 20% | 1636 |
| 40% | 2168 |
| 60% | 5691 |
| 80% | 8655 |
| 100% | 14979 |

4-5. Confirmation of Removal of Noise from MRI Image Using Glycerin

According to the method described in Example 1-1, a pad was manufactured by filling a pad with glycerin (C₃H₈O₃), and an MRI image was measured.

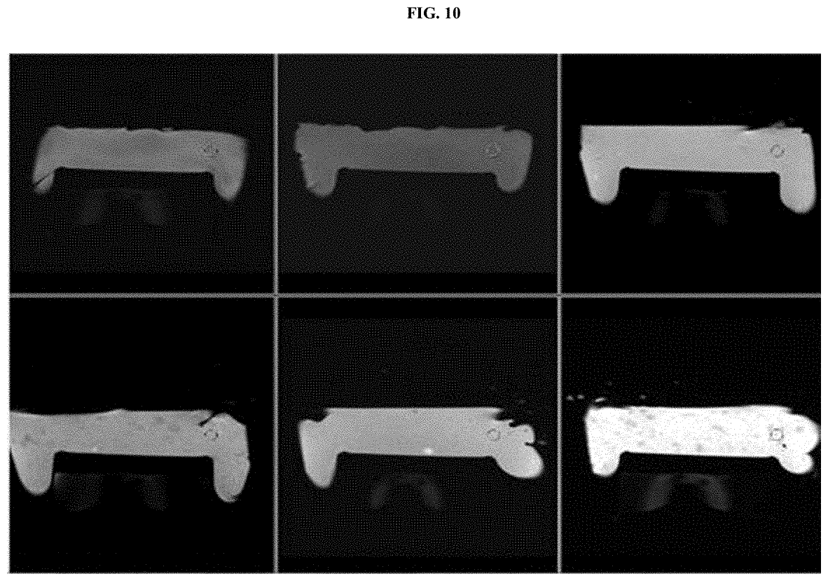

As a result, as shown in FIG. 10 and Table 7, it can be observed that, from the left side to the right side of FIG. 10 (the concentration of glycerin in the upper panels was sequentially increased to 20% and 40% from 0% (H₂O), and similarly, the concentration of glycerin in the lower panels was sequentially increased to 60%, 80% and 100% from the left side to the right side of the drawing), as the concentration of glycerin was gradually increased, the ROI mean value was increased, and the noise reduction rate of an MRI image was increased.

TABLE 7

| Concentration of glycerin (C₃H₈O₃) | ROI mean value (Mean) |
|---|---|
| 0% (H₂O) | 1397 |
| 20% | 1488 |
| 40% | 1503 |
| 60% | 1538 |
| 80% | 1702 |
| 100% | 1712 |

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

The present invention relates to a composition for removing MRI noise using a compound having a functional group capable of hydrogen bonding and a pad using the same, and the composition or pad according to the present invention reduces noise caused by a metal implant in the acquisition of MRI images, has an excellent image enhancement effect, is cheap and economical, has no toxicity in a human body and thus is safe. For this reason, the composition can be used as a component of an image diagnostic device in the field of medical devices, and is expected to be effectively used to acquire noise-removed MRI images in the field of diagnostic radiology.

1. A composition for removing noise from an MRI image, comprising:
a compound having a functional group capable of hydrogen bonding.

2. The composition according to claim 1, wherein the composition improves the quality of an MRI image.

3. The composition according to claim 1, wherein the compound is one or more selected from the group consisting of water (H₂O), amyl alcohol (C₅H₁₁OH), butyl alcohol (CH₃CH₂CH₂OH), ethanol (C₂H₅OH), methyl alcohol (CH₃OH), ethylene glycol (OHCH₂CH₂OH), formaldehyde (HCHO), nitrobenzene (C₆H₅NO₂), propylene glycol (OH(CH₂)₃H), hydrogen peroxide (H₂O₂), acetic acid (CH₃COOH), acetic anhydride (C₄H₆O₃), butanic acid (C₃H₇COOH), benzenesulfonic acid (C₆H₅SO₃H), benzoic acid (C₆H₅COOH), chromic acid (H₂CrO₄), citric acid (C₆H₈O₇H₂O), hexafluorosilicic acid (H₂SiF₆), fluoroboric acid (HBF₄), formic acid (HCOOH), hydrochloric acid (HCl), hydrocyanic acid (HCN), hydrofluoric acid (HF), hypochlorous acid (HClO), lactic acid (CH₃CHOHCOOH), maleic acid ((CHOOH)₂), nitric acid (HNO₃), oleic acid (C₁₈H₃₄O₂), phosphoric acid (H₃PO₄), perchloric acid (HClO₄), sulfuric acid (H₂SO₄), stearic acid (C₁₇H₃₅COOH), trichloroacetic acid (CCl₃COOH), ammonia (NH₄OH), sodium hydrogen carbonate (NaHCO₃), ammonium carbonate ((NH₄)₂CO₃), ammonium chloride ((NH₄)₂Cl), ammonium nitride (NH₄NO₃), ammonium persulfate ((NH₄)₂S₂O₈), glycerin (C₃H₈O₃), ammonium sulfate ((NH4)₂SO₄) and ammonium bicarbonate (NH₄HCO₃).

4. The composition according to claim 1, wherein the noise is generated by a metal inserted into the body.

5. A pad for removing noise from an MRI image, comprising the composition of claim 1.

6. The pad according to claim 5, which improves the quality of an MRI image.

7. The pad according to claim 5, further comprising a cover accommodating the composition.

8. The pad according to claim 5, which is transformable into a shape of the human body.

9. The pad according to claim 5, which is attachable/detachable to a main body coil, a shim coil, an RF coil, an array coil, a surface coil, a spine coil, a head coil, an extremity coil or a torso coil for an MRI apparatus.

10. A coil for an MRI apparatus into which the composition of claim 1 is injected.

11. The coil according to claim 10, which is one or more selected from the group consisting of a main body coil, a shim coil, an RF coil, an array coil, a surface coil, a spine coil, a head coil, an extremity coil or a torso coil for an MRI apparatus.

12. A method of removing noise from an MRI image using a composition comprising a compound having a functional group capable of hydrogen bonding.

13. The method according to claim 12, wherein the compound is one or more selected from the group consisting of water (H₂O), amyl alcohol (C₅H₁₁OH), butyl alcohol (CH₃CH₂CH₂H), ethanol (C₂H₅OH), methyl alcohol (CH₃OH), ethylene glycol (OHCH₂CH₂OH), formaldehyde (HCHO), nitrobenzene (C₆H₅NO₂), propylene glycol (OH(CH₂)₃H), hydrogen peroxide (H₂O₂), acetic acid (CH₃COOH), acetic anhydride (C₄H₆O₃), butanic acid (C₃H₇COOH), benzenesulfonic acid (C₆H₅SO₃H), benzoic acid (C₆H₅COOH), chromic acid (H₂CrO₄), citric acid (C₆H₈O₇H₂O), hexafluorosilicic acid (H₂SiF₆), fluoroboric acid (HBF₄), formic acid (HCOOH), hydrochloric acid (HCl), hydrocyanic acid (HCN), hydrofluoric acid (HF), hypochlorous acid (HClO), lactic acid (CH₃CHOHCOOH), maleic acid ((CHOOH)₂), nitric acid (HNO₃), oleic acid (C₁₈H₃₄O₂), phosphoric acid (H₃PO₄), perchloric acid (HClO₄), sulfuric acid (H₂SO₄), stearic acid (C₁₇H₃₅COOH), trichloroacetic acid (CCl₃COOH), ammonia (NH₄OH), sodium hydrogen carbonate (NaHCO₃), ammonium carbonate ((NH₄)₂CO), ammonium chloride ((NH₄)₂Cl), ammonium nitride (NH₄NO₃), ammonium persulfate ((NH₄)₂S₂O₈), glycerin (C₃H₈O₃), ammonium sulfate ((NH4)₂SO₄) and ammonium bicarbonate (NH₄HCO₃).

14. A method of obtaining a noise-reduced MRI image, comprising:
manufacturing a pad including a compound having a functional group capable of hydrogen bonding;
attaching the pad to a desired region of a subject; and
obtaining an MRI image in the desired region using an MRI apparatus.

15. The method according to claim 14, wherein the compound is one or more selected from the group consisting of water (H₂O), amyl alcohol (C₅H₁₁OH), butyl alcohol (CH₃CH₂CH₂OH), ethanol (C₂H₅OH), methyl alcohol (CH₃OH), ethylene glycol (OHCH₂CH₂OH), formaldehyde (HCHO), nitrobenzene (C₆H₅NO₂), propylene glycol (OH(CH₂)₃OH), hydrogen peroxide (H₂O₂), acetic acid (CH₃COOH), acetic anhydride (C₄H₆O₃), butanic acid (C₃H₇COOH), benzenesulfonic acid (C₆H₅SO₃H), benzoic acid (C₆H₅COOH), chromic acid (H₂CrO₄), citric acid (C₆H₈O₇H₂O), hexafluorosilicic acid (H₂SiF₆), fluoroboric acid (HBF₄), formic acid (HCOOH), hydrochloric acid (HCl), hydrocyanic acid (HCN), hydrofluoric acid (HF), hypochlorous acid (HClO), lactic acid (CH₃CHOHCOOH), maleic acid ((CHOOH)₂), nitric acid (HNO₃), oleic acid (C₁₈H₃₄O₂), phosphoric acid (H₃PO₄), perchloric acid (HClO₄), sulfuric acid (H₂SO₄), stearic acid (C₁₇H₃₅COOH), trichloroacetic acid (CCl₃COOH), ammonia (NH₄OH), sodium hydrogen carbonate (NaHCO₃), ammonium carbonate ((NH₄)₂CO₃), ammonium chloride ((NH₄)₂Cl), ammonium nitride (NH₄NO₃), ammonium persulfate ((NH₄)₂S₂O₈), glycerin (C₃H₈O₃), ammonium sulfate ((NH4)₂SO₄) and ammonium bicarbonate (NH₄HCO₃).

16. (canceled)
